(19) United States
(12) Patent Application Publication
(10) Pub. No.: US 2023/0029947 A1
(43) Pub. Date: Feb. 2, 2023

(54) MEDICAL DISEASE FEATURE SELECTION METHOD BASED ON IMPROVED SALP SWARM ALGORITHM

(71) Applicant: Wenzhou University, Zhejiang (CN)

(72) Inventors: Pengjun Wang, Zhejiang (CN); Songwei Zhao, Zhejiang (CN); Huiling Chen, Zhejiang (CN); Suling Xu, Zhejiang (CN); Wenming He, Zhejiang (CN); Yijian Shi, Zhejiang (CN)

(73) Assignee: Wenzhou University, Zhejiang (CN)

(21) Appl. No.: 17/860,077

(22) Filed: Jul. 7, 2022

(30) Foreign Application Priority Data
Jul. 23, 2021 (CN) 202110834402.1

(51) Int. Cl. G06N 3/12; G06K 9/62; G16H 10/40; G16H 50/20

(52) U.S. Cl. CPC G06N 3/126; G06K 9/6251; G16H 10/40; G16H 50/20

(57) ABSTRACT

The disclosure is a medical disease feature selection method based on an improved salp swarm algorithm. The improved salp swarm algorithm is used to optimize a feature selection problem, the accuracy of the mentioned method is estimated by means of a transfer function and classification by a K-nearest neighbor algorithm, and the salp swarm algorithm is improved by using a self-adapted control parameter and an elite grey wolf ruling policy, so that it helps the algorithm avoid premature convergence in the optimization process and jump out of local optimum, thereby achieving a target of the algorithm with the smallest selected feature quantity and the highest classification precision. The method has the advantages of high rate of convergence, higher classification precision and better robustness.

### CROSS-REFERENCE TO RELATED APPLICATION

This application claims the priority benefit of China application serial no. 202110834402.1 filed on Jul. 23, 2021. The entirety of the above-mentioned patent application is hereby incorporated by reference herein and made a part of this specification.

### BACKGROUND

### Technical Field

The present invention relates to a feature selection method for medical diseases, in particular to a medical disease feature selection method based on an improved salp swarm algorithm.

### Description of Related Art

With wide application of gene chip technology in the medical field, a lot of microarray data is accumulated quickly. By analyzing the data and constructing an effective classification model, it is of important research meaning and application value to early diagnosis and clinical treatment of some potential diseases. However, a microarray gene date set has the characteristic of high-dimensional small samples, for example, a breast cancer microarray gene date set includes more than two thousand gene features. In the face of such a large scale microarray gene data set, an expert cannot perform analysis and diagnostic treatment directly within a short time. In addition, most gene data usually contains some redundancy or noise data. The information may greatly reduce the performance of sorting algorithm learning, so that the efficiency is reduced and the medical diagnosis will be affected. As an effective dimensionality-reducing way, feature selection has received wide attention in the biomedical field and became the research spot in the field of bioinformatics in recent years. A feature selection technology is a critical step to analyze and classify the microarray gene data set properly. If there are no proper feature selection methods, it is hard for an existing classification model to capture important information. Essentially, as a typical global optimization problem, the feature selection problem is one of the most important links. Different from other dimensionality-reducing technologies, feature selection does not change original representation of variable features but selects subsets thereamong. Therefore, feature selection reserves the original variable condition, so that it has the advantage of further explaining feature data. In addition, complexity and prediction effect of the sorting algorithm are closely linked to sample features. Redundancy and relevance of sample features will decrease prediction ability, and meanwhile, amplitude of feature dimension also affects the operating velocity of the sorting algorithm.

Feature selection is essentially a combined optimization problem. A conventional optimization algorithm such as an analytical method requires a continuous and finely adjustable target function, and the obtained optimal solution does not reach the needed precision usually. Although an enumeration method overcomes the deficiencies, the calculating efficiency is too low. Even the considerably famous dynamic planning method will come across an exponential explosion problem, and it often shows weakness for problems with medium scales and proper complexity. Therefore, if the optimization ability of an swarm intelligence algorithm can be creatively applied to the feature selection problem, it will provide a powerful explaining tool for analysis of medical disease features.

At present, there have been much research applying the swarm intelligence algorithm to search for feature subsets in an assisted manner, and remarkable effects have been achieved. The Salp Swarm Algorithm (SSA) is an emerging heuristic swarm intelligence algorithm. Inspired by a salp foraging process, it includes three stages: approaching food, wrapping food and searching for food, which realizes continuous exploration and development of the whole searching space.

However, SSA still has phenomena of local optimization and premature convergence in the process of searching for the feature subsets, thereby, finally leading to reduction of the selection accuracy of the feature subsets.

Therefore, it is necessary to provide an improved salp swarm algorithm which can solve the problems of a locally optimal solution, low rate of convergence and the like of the SSA, thereby achieving more precise and efficient classification and prediction of medical disease features.

### SUMMARY

The present invention aims to provide a medical disease feature selection method based on an improved salp swarm algorithm with higher classification precision and better robustness.

A technical scheme adopted by the present invention to solve the above-mentioned technical problems is as follows: a medical disease feature selection method based on an improved salp swarm algorithm includes the following steps:

Step S1, acquiring a microarray gene data set of medical diseases, marking a line number of the microarray gene data set of medical diseases as m and a column number thereof as n, wherein the obtained microarray gene data set of medical diseases is formed by arranging m*n gene feature data according to m lines and n columns; partitioning the microarray gene data set of medical diseases into 10 subsets randomly by using a 10-cross validation function, wherein the line number of each subset is greater than or equal to 1 and the column numbers are n; and selecting one subset from the 10 subsets as a validation set, the rest of subsets being training sets;

Step S2, defining a female salp population Y, wherein a size of the female salp population Y being M=20, i.e., there are M individuals in the female salp population Y, and each individual in the female salp population Y is respectively represented by a data matrix formed by arranging n dimensionality values in one line and n columns; and then performing initialized assignment to each dimensionality value of each individual in the female salp population Y by using a random number between 0 and 1 to obtain the 0th female salp population Y⁰;

Step S3, setting the global optimum fitness value is best, performing initialized assignment of best to positive infinity, setting the global optimal individual to be bestposition, and initially setting the bestposition as a data matrix [0, 0, 0, . . . , 0] with one line and n columns;

Step S4, setting the maximum time of iteration of female salp population T=50, setting an iterative time variable t and initially setting t as 1;

Step S5, performing the t^{th} iteration on the female salp population, the iteration specifically including:

Step S5.1, converting each dimensionality value of each individual in the (t-1)^{th} generation female salp population Y^{t-1} into 0 or 1 via transfer functions shown in formulae (1)-(2) to obtain a t^{th} generation binary salp population B^{t};

$${B}_{i , j}^{t} = \{ \begin{matrix}1 , & S ( {Y}_{i , j}^{t - 1} ) \geq r \\ 0 , & S ( {Y}_{i , j}^{t - 1} ) < r\end{matrix} \qquad ( 1 )$$

$$S ( {Y}_{i , j}^{t - 1} ) = \frac{1}{1 + {e}^{- ( {Y}_{i , j}^{t - 1} / 3 )}} , \qquad ( 2 )$$

wherein Y_{i,j}^{t-1}represents a dimensionality value in the j^{th} column of the i^{th} individual in the (t−1)^{th} generation female salp population, i is equal to 1, 2, 3, . . . M, j is equal to 1, 2, 3, . . . n, Bₜ represents a dimensionality value in the j^{th} column of the i^{th} individual in the th generation binary salp population, r is a random number between 0 and 1 and is generated a random function before operation every time, and e is a natural constant;

Step S5.2, constructing a feature subset of each individual in the (t−1)^{th} generation female salp population, the step specifically including: judging whether the dimensionality value of each column in the i^{th} individual in the t^{th} generation binary salp population is 1 or not respectively, if it is 1, selecting gene feature data in a verification set and 9 training sets located in the column, and if it is 0, not selecting the gene feature data in a verification set and 9 training sets located in the column, taking the residual part as the feature subset of the verification set of the i^{th} individual in the (t−1)^{th} generation female salp population after deleting the gene feature data of all the unselected columns in the verification set, and taking the residual part as the feature subsets of the 9 training sets of the i^{th} individual in the (t−1)^{th} generation female salp population after deleting the gene feature data of all the unselected columns in the 9 training sets, thereby obtaining the feature subset of the verification set of each individual in the (t−1)^{th} generation female salp population and the feature subsets of the 9 training sets;

Step S5.3, calculating a fitness value of each individual in the (t−1)^{th} generation female salp population by adopting a formula (3) and a formula (4), sequencing all the individuals in the (t−1)^{th} generation female salp population according to the fitness values from small to large, marking the minimum fitness as bF^{t-1}, and taking the individual with the minimum fitness value as a current optimum individual marked as bP^{t-1};

$$f i {t}_{i}^{t - 1} = ( 1 - a c {c}_{i}^{t} ) \times a + \frac{{L}_{i}^{t}}{n} \times b \qquad ( 3 )$$

$${acc}_{i}^{t} = \frac{c {c}_{i}^{t}}{c {c}_{i}^{t} + u {c}_{i}^{t}} \times 1 \; 00 \% , \qquad ( 4 )$$

wherein fitᵢ^{t-1}represents a fitness value of the i^{th} individual in the (t−1)^{th} generation female salp population, a represents a classification accuracy weight set as 0.05, b represents the optimum feature selection number weight, a relation between a and b is a+b=1, Lt represents a total column number with the dimensionality value of 1 in the i^{th} individual in the (t−1)^{th} generation female salp population, acct represents a classification accuracy of the i^{th} individual obtained by the K-nearest algorithm, ccᵢ^{t} and ucᵢ^{t} are obtained by performing classifying statistical test on data in the feature subset of the verification set and data in the feature subsets of the 9 training sets in the i^{th} individual in the (t−1)^{th} generation female salp population by adopting the K-nearest algorithm, ccᵢ^{t} represents a number of corrected classified data in the feature subset of the verification set and ucᵢ^{t} represents a number of mistaken classified data in the feature subset of the verification set;

Step S5.4, updating the dimensionality values from the first individual to the M/2th individual in the t^{th} generation binary salp population B^{t} by adopting a formula (5) respectively to obtain the first individual to the M/2^{th} individual of the t^{th} generation binary salp population F^{t};

$${F}_{k , j}^{t} = \{ \begin{matrix}b {P}_{j}^{t - 1} + {c}^{t} r {1}^{t} r {2}^{t} \geq 0 \\ b {P}_{j}^{t - 1} - {c}^{t} r {1}^{t} r {2}^{t} < 0\end{matrix} \qquad ( 5 )$$

$${c}^{t} = 2 {e}^{- {( \frac{4 * t}{T} )}^{2}} , \qquad ( 6 )$$

wherein k is equal to 1, 2, 3 . . . , M/2, r1^{t} and r2^{t} is respectively a random number generated by a random function between 0 and 1, c^{t} is a control parameter represented by a formula (6), bPⱼ^{t-1} represents a current optimum individual the j^{th} column dimensionality value of bP^{t-1}, Ft represents the k^{th} individual the j^{th} column dimensionality value in the t^{th} generation initial salp population Ft and e is a natural constant;

Step S5.5, updating the dimensionality values from the (M/2+1)^{th} individual to the “^{h}individual in the t^{th} generation binary salp population B^{t} by adopting a formula (7) respectively by means of the self-adapted control parameter to obtain the (M/2+1)^{th} individual to the M^{th} individual of the t^{th} generation binary salp population F^{t}:

$${F}_{d}^{t} = \frac{1}{2} \times cos ( \frac{p i \times t}{2 T} ) \times ( {B}_{d}^{t} + {B}_{d - 1}^{t} ) \qquad ( 7 )$$

wherein d is equal to M/2+1, M/2+2, M/2+3, . . . , M, B_{d} ^{t} represents the d^{th} individual of the t^{th} generation binary salp population B^{t}, B_{d-1} ^{t} represents the (d−1)^{th} individual in the t^{th} generation binary salp population B^{t}, F_{d}^{t} represents the d^{th} individual in the t^{th} generation initial salp population F^{t}, pi refers to circularity ratio and cos represents a cosine function;

Step S5.6, calculating the fitness value of each individual in the t^{th} generation initial salp population F^{t} by adopting a method same with those in the Steps 5.1-5.3, sequencing all the individuals in the t^{th} generation initial salp population F^{t} according to the fitness values from small to large, and marking the individual with the minimum fitness value as fir^{t}, the individual with the secondary minimum fitness value as sect and the individual with the third minimum fitness value as thi^{t};

Step S5.7, exploring and developing the t^{th} generation initial salp population Ft by adopting formulae (8)-(16) based on the elite grey wolf ruling policy to obtain the t^{th} generation intermediate salp population G^{t};

$$d i {s}_{j}^{f i r , t} = \vert 2 r {3}^{t} \times f i {r}_{j}^{t} - {F}_{i , j}^{t} \vert \qquad ( 8 )$$

$${G}_{i , j}^{f i r , t} = f i {r}_{j}^{t} - {A}^{t} \times d i {s}_{j}^{f i r , t} \qquad ( 9 )$$

$${A}^{t} = 2 {β}^{t} \times r {4}^{t} - {β}^{t} \qquad ( 10 )$$

$${β}^{t} = 2 - \frac{2 t}{T} \qquad ( 11 )$$

$$di {s}_{j}^{s e c , t} = \vert 2 r {3}^{t} \times s e {c}_{j}^{t} - {F}_{i , j}^{t} \vert \qquad ( 12 )$$

$${G}_{i , j}^{s e c , t} = s e {c}_{j}^{t} - {A}^{t} \times d i {s}_{j}^{s e c , t} \qquad ( 13 )$$

$$di {s}_{j}^{t h i , t} = \vert 2 r {3}^{t} \times t h {i}_{j}^{t} - {F}_{i , j}^{t} \vert \qquad ( 14 )$$

$${G}_{i , j}^{t h i , t} = t h {i}_{j}^{t} - {A}^{t} \times d i {s}_{j}^{t h i , t} \qquad ( 15 )$$

$${G}_{i , j}^{t} = \frac{{G}_{i , j}^{f i r , t} + {G}_{i , j}^{s e c , t} + {G}_{i , j}^{t h i , t}}{3} , \qquad ( 16 )$$

wherein r3^{t} and r4^{t} are respectively random numbers generated by the random function between 0 and 1, At and f₃^{t} are vector coefficients, firit represents the j^{th} column dimensionality value of the individual with the minimum fitness value in the t^{th} generation initial salp population F^{t}, secⱼ ^{t} represents the j^{th} column dimensionality value of the individual with the secondary minimum fitness value in the t^{th} generation initial salp population F^{t}, thiⱼ^{t} represents the j^{th} column dimensionality value of the individual with the third minimum fitness value in the t^{th} generation initial salp population F^{t}, F_{i,j}^{t} represents the j^{th} column dimensionality value of the i^{th} individual in the t^{th} generation initial salp population Ft, and G_{i,j} ^{t} represents the j^{th} column dimensionality value of the i^{th} individual in the t^{th} generation intermediate salp population G^{t};

Step S5.8, calculating the fitness value of the t^{th} generation intermediate salp population Gt by adopting the method same with those in the Steps S5.1-5.3, combining the M individuals in the t^{th} generation initial salp population Ft and the M individuals in the t^{th} generation intermediate salp population Gt, sequencing the totally 2M individuals according to the fitness values from small to big, selecting the M individuals with smaller fitness values, and arranging the M individuals randomly as the t^{th} iteration to obtain the t^{th} generation salp population Y^{t}; and

Step S5.9, comparing the minimum fitness value of the t^{th} generation salp population Y^{t} with the global optimum fitness value best, updating best by adopting the minimum fitness value if the minimum fitness value is smaller than the global optimum fitness value best and taking the individual corresponding to the minimum fitness value as the global optimum individual bestposition, and keeping the global optimum individuals bestposition with the global optimum fitness value best invariable if the minimum fitness value is not smaller than the global optimum fitness value best and finishing the t^{th} iteration; and

Step S6, judging whether the current value of t is equal to T or not, if not, updating the value of t by adopting the current value of t plus 1, and then returning to the Step S5 for next iteration; if it is equal to T, finishing the iteration process, determining columns with the dimensionality value of 1 from the first column to the nth column of the current global optimum individual bestposition, and extracting the gene feature data in the microarray gene data set of the medical diseases in these columns correspondingly to form a selection data set, wherein the selection data set obtained at the time is the dimensionality-reducing gene feature data set of the medical diseases.

Compared with the prior art, the present invention has the advantages that the improved salp swarm algorithm is used to optimize a feature selection problem, the accuracy of the mentioned method is estimated by means of a transfer function and classification by a K-nearest neighbor algorithm, and the salp swarm algorithm is improved by using a self-adapted control parameter and an elite grey wolf ruling policy, so that it helps the algorithm avoid premature convergence in the optimization process and jump out of local optimum, thereby achieving a target of the algorithm with the smallest selected feature quantity and the highest classification precision.

Thus, the method of the present invention has the advantages of high rate of convergence, higher classification precision and better robustness.

### DESCRIPTION OF THE EMBODIMENTS

Further description of the present invention in detail will be made below in combination with embodiments.

Embodiment: a medical disease feature selection method based on an improved salp swarm algorithm, including the following steps:

Step S1, a microarray gene data set of medical diseases is acquired, a line number of the microarray gene data set of medical diseases is marked as m and a column number thereof as n, wherein the obtained microarray gene data set of medical diseases is formed by arranging m*n gene feature data according to m lines and n columns; the microarray gene data set of medical diseases is partitioned into 10 subsets randomly by using a 10-cross validation function, wherein the line number of each subset is greater than or equal to 1 and the column numbers are n; and selecting one subset from the 10 subsets as a validation set, the rest of subsets being training sets;

Step S2, a female salp population Y is defined, wherein a size of the female salp population Y being M=20, i.e., there are M individuals in the female salp population Y, and each individual in the female salp population Y is respectively represented by a data matrix formed by arranging n dimensionality values in one line and n columns; and then initialized assignment performed to each dimensionality value of each individual in the female salp population Y by using a random number between 0 and 1 to obtain the 0^{th} female salp population Y⁰;

Step S3, the global optimum fitness value is set as best, initialized assignment of best is performed to positive infinity, the global optimal individual is set to be bestposition, and initially setting the bestposition as a data matrix [0, 0, 0, . . . , 0] with one line and n columns;

Step S4, the maximum time of iteration of female salp population is set at T=50, an iterative time variable t is set and t is initially set as 1;

Step S5, the t^{th} iteration is performed on the female salp population, the iteration specifically including:

Step S5.1, each dimensionality value of each individual in the (t−1)^{th} female salp population Y^{t-1} is converted into 0 or 1 via transfer functions shown in formulae (1)-(2) to obtain a t^{th} binary salp population B^{t};

$${B}_{i , j}^{t} = \{ \begin{matrix}1 , & S ( {Y}_{i , j}^{t - 1} ) \geq r \\ 0 , & S ( {Y}_{i , j}^{t - 1} ) < r\end{matrix} , \qquad ( 1 )$$

$$S ( {Y}_{i , j}^{t - 1} ) = \frac{1}{1 + {e}^{- ( {Y}_{i , j}^{t - 1} / 3 )}} , \qquad ( 2 )$$

wherein Y_{i,j}^{t-1} represents a dimensionality value in the j^{th} column of the i^{th} individual in the (t−1)^{th} female salp population, i is equal to 1, 2, 3, . . . M, j is equal to 1, 2, 3, . . . n, B represents a dimensionality value in the j^{th} column of the i^{th} individual in the t^{th} binary salp population, r is a random number between 0 and 1 and is generated a random function before operation every time, and e is a natural constant;

Step S5.2, a feature subset of each individual in the (t−1)^{th} generation female salp population is constructed, the step specifically including: whether the dimensionality value of each column in the i^{th} individual in the t^{th} generation binary salp population is 1 or not judged respectively, if it is 1, gene feature data in a verification set and 9 training sets located in the column is selected, and if it is 0, the gene feature data in a verification set and 9 training sets located in the column is not selected, the residual part is taken as the feature subset of the verification set of the i^{th} individual in the (t−1)^{th} generation female salp population after deleting the gene feature data of all the unselected columns in the verification set, and the residual part is taken as the feature subsets of the 9 training sets of the i^{th} individual in the (t−1)^{th} generation female salp population after deleting the gene feature data of all the unselected columns in the 9 training sets, thereby obtaining the feature subset of the verification set of each individual in the (t−1)^{th} generation female salp population and the feature subsets of the 9 training sets;

Step S5.3, a fitness value of each individual in the (t−1)^{th} generation female salp population is calculated by adopting a formula (3) and a formula (4), all the individuals in the (t−1)^{th} generation female salp population are sequenced according to the fitness values from small to large, the minimum fitness is marked as bF^{t-1}, and the individual with the minimum fitness value is taken as a current optimum individual marked as bP^{t-1};

$$f i {t}_{i}^{t - 1} = ( 1 - a c {c}_{i}^{t} ) \times a + \frac{{L}_{i}^{t}}{n} \times b , \qquad ( 3 )$$

$${acc}_{i}^{t} = \frac{c {c}_{i}^{t}}{c {c}_{i}^{t} + u {c}_{i}^{t}} \times 1 \; 00 \% , \qquad ( 4 )$$

wherein fit -i represents a fitness value of the i^{th} individual in the (t−1)^{th} generation female salp population, a represents a classification accuracy weight set as 0.05, b represents the optimum feature selection number weight, a relation between a and b is a+b =1, Lᵢ^{t} represents a total column number with the dimensionality value of 1 in the i^{th} individual in the (t−1)^{th} generation female salp population, accᵢ^{t} represents a classification accuracy of the i^{th} individual obtained by the K-nearest algorithm, ccᵢ^{t} and ucᵢ^{t} are obtained by performing classifying statistical test on data in the feature subset of the verification set and data in the feature subsets of the 9 training sets in the i^{th} individual in the (t−1)^{th} generation female salp population by adopting the K-nearest algorithm, cc[represents a number of corrected classified data in the feature subset of the verification set and uct represents a number of mistaken classified data in the feature subset of the verification set;

Step S5.4, the dimensionality values from the first individual to the M/2^{th} individual in the t^{th} generation binary salp population Bt are updated by adopting a formula (5) respectively to obtain the first individual to the M/2^{th} individual of the t^{th} generation binary salp population F^{t};

$${F}_{k , j}^{t} = \{ \begin{matrix}b {P}_{j}^{t - 1} + {c}^{t} r {1}^{t} r {2}^{t} \geq 0 \\ b {P}_{j}^{t - 1} - {c}^{t} r {1}^{t} r {2}^{t} < 0\end{matrix} \qquad ( 5 )$$

$${c}^{t} = 2 {e}^{- {( \frac{4 * t}{T} )}^{2}} \qquad ( 6 )$$

wherein k is equal to 1, 2, 3 . . . , M/2, r1^{t} and r2^{t} is respectively a random number generated by a random function between 0 and 1, ct is a control parameter represented by a formula (6), bPit-1 represents a current optimum individual the j^{th} column dimensionality value of bPt-1, Fₜ represents the k^{th} individual the j^{th} column dimensionality value in the t^{th} generation initial salp population Ft and e is a natural constant;

Step S5.5, the dimensionality values from the (M/2+1)^{th} individual to the M^{th} individual in the t^{th} generation binary salp population B^{t} are updated by adopting a formula (7) respectively by means of the self-adapted control parameter to obtain the (M/2+1)^{th} individual to the M^{th} 10 individual of the t^{th} generation binary salp population Ft:

$${F}_{d}^{t} = \frac{1}{2} \times cos ( \frac{p i \times t}{2 T} ) \times ( {B}_{d}^{t} + {B}_{d - 1}^{t} ) \qquad ( 7 )$$

wherein d is equal to M/2+1, M/2+2, M/2+3, . . . , M, B_{d}^{t} represents the d^{th} individual of the t^{th} generation binary salp population B^{t}, B_{d-1} ^{t} represents the (d−1)^{th} individual in the t^{th} generation binary salp population B^{t}, Fd represents the dth individual in the t^{th} generation initial salp population F^{t}, pi refers to circularity ratio and cos represents a cosine function; 15 [0053] Step S5.6, the fitness value of each individual in the t^{th} generation initial salp population Ft is calculated by adopting a method same with those in the Steps 5.1-5.3, all the individuals in the t^{th} generation initial salp population Ft are sequenced according to the fitness values from small to large, and the individual with the minimum fitness value is marked as firt, the individual with the secondary minimum fitness value as sect and the individual with the third minimum fitness value as thi^{t};

Step S5.7, the t^{th} generation initial salp population Ft is explored and developed by adopting formulae (8)-(16) based on the elite grey wolf ruling policy to obtain the t^{th} generation intermediate salp population Gt;

$$d i {s}_{j}^{f i r , t} = \vert 2 r {3}^{t} \times f i {r}_{j}^{t} - {F}_{i , j}^{t} \vert \qquad ( 8 )$$

$${G}_{i , j}^{f i r , t} = f i {r}_{j}^{t} - {A}^{t} \times d i {s}_{j}^{f i r , t} \qquad ( 9 )$$

$${A}^{t} = 2 {β}^{t} \times r {4}^{t} - {β}^{t} \qquad ( 10 )$$

$${β}^{t} = 2 - \frac{2 t}{T} \qquad ( 11 )$$

$$di {s}_{j}^{s e c , t} = \vert 2 r {3}^{t} \times s e {c}_{j}^{t} - {F}_{i , j}^{t} \vert \qquad ( 12 )$$

$${G}_{i , j}^{s e c , t} = s e {c}_{j}^{t} - {A}^{t} \times d i {s}_{j}^{s e c , t} \qquad ( 13 )$$

$$di {s}_{j}^{t h i , t} = \vert 2 r {3}^{t} \times t h {i}_{j}^{t} - {F}_{i , j}^{t} \vert \qquad ( 14 )$$

$${G}_{i , j}^{t h i , t} = t h {i}_{j}^{t} - {A}^{t} \times d i {s}_{j}^{t h i , t} \qquad ( 15 )$$

$${G}_{i , j}^{t} = \frac{{G}_{i , j}^{f i r , t} + {G}_{i , j}^{s e c , t} + {G}_{i , j}^{t h i , t}}{3} \qquad ( 16 )$$

wherein r3^{t} and r4^{t} are respectively random numbers generated by the random function between 0 and 1, A^{t} and β^{t} are vector coefficients, firⱼ^{t} represents the j^{th} column dimensionality value of the individual with the minimum fitness value in the t^{th} generation initial salp population F^{t}, secⱼ^{t} represents the j^{th} column dimensionality value of the individual with the secondary minimum fitness value in the t^{th} generation initial salp population F^{t}, thiⱼ^{t} represents the j^{th} column dimensionality value of the individual with the third minimum fitness value in the t^{th} generation initial salp population Ft, _{F} represents the j^{th} column dimensionality value of the i^{th} individual in the t^{th} generation initial salp population Ft, and Giₜ represents the j^{th} column dimensionality value of the i^{th} individual in the t^{th} generation intermediate salp population Gt;

Step S5.8, the fitness value of the t^{th} generation intermediate salp population Gt is calculated by adopting the method same with those in the Steps S5.1-5.3, the M individuals in the th generation initial salp population F^{t} and the M individuals in the t^{th} generation intermediate salp population G^{t} are combined, the totally 2M individuals are sequenced according to the fitness values from small to big, the M individuals with smaller fitness values are selected, and the M individuals randomly are arranged as the t^{th} iteration to obtain the t^{th} generation salp population Y^{t}; and

Step S5.9, the minimum fitness value of the t^{th} generation salp population yt is compared with the global optimum fitness value best, best is updated by adopting the minimum fitness value if the minimum fitness value is smaller than the global optimum fitness value best, and the individual corresponding to the minimum fitness value is taken as the global optimum individual bestposition, and it is kept that the global optimum individuals bestposition with global optimum fitness value best invariable if the minimum fitness value is not smaller than the global optimum fitness value best, and finishing the t^{th} iteration; and

Step S6, whether the current value oft is equal to T or not is judged, if not, the value oft is updated by adopting the current value of t plus 1, and then it is returned to the Step S5 for next iteration; if it is equal to T, the iteration process is finished, columns with the dimensionality value of 1 from the first column to the nth column of the current global optimum individual bestposition are determined, and the gene feature data in the microarray gene data set of the medical diseases in these columns are extracted correspondingly to form a selection data set, wherein the selection data set obtained at the time is the dimensionality-reducing gene feature data set of the medical diseases.

By taking four data sets D1-D4 in a UCI machine learning library as an example, comparative analysis is performed by adopting the method of the present invention and an existing slap swarm algorithm, where specific information of the four data sets D1-D4 is as shown in a table 1. Results of the fitness values respectively obtained by the method (AGSSA) of the present invention and the existing SSA are shown in a table 2. When the fitness value is minimum, the selected feature quantity is as shown in a table 3. When the fitness value is minimum, an error rate of the feature quantity selected based on the K-nearest algorithm is as shown in a table 4:

TABLE 1

| No. | Name | No. of Instances | No. of Features |
|---|---|---|---|
| D1 | Exactly | 1000 | 14 |
| D2 | Lymphography | 148 | 19 |
| D3 | Vote | 300 | 17 |
| D4 | WineEW | 178 | 14 |

TABLE 2

| | AGSSA | SSA |
|---|---|---|
| D1 | 0.0300 | 0.0376 |
| D2 | 0.0357 | 0.0465 |
| D3 | 0.0243 | 0.0321 |
| D4 | 0.0126 | 0.0154 |

TABLE 3

| | AGSSA | SSA |
|---|---|---|
| D1 | 7.15 | 7.70 |
| D2 | 6.36 | 7.54 |
| D3 | 3.73 | 5.31 |
| D4 | 3.28 | 4.01 |

TABLE 4

| | AGSSA | SSA |
|---|---|---|
| D1 | 0.0026 | 0.0084 |
| D2 | 0.0190 | 0.0269 |
| D3 | 0.0133 | 0.0163 |
| D4 | 0.0000 | 0.0000 |

It may be seen from the above data that on the four data sets, the fitness value of the method of the present invention is minimum, indicating that the method has better optimal performance on feature selection problem. It may be seen from feature quantity selection that on the four data sets, the quantity selected by the improved salp swarm algorithm provided by the present invention is smaller than that of an original salp swarm algorithm, indicating that improvement on the algorithm is effectively, thereby helping the algorithm jumping out of local optimization and increasing the probability of finding the optimal solution. It may be seen from the error rate data that the feature selection classification error rate of the method of the present invention is also smaller than that of the original SSA, which reflects superiority of the algorithm provided by the present invention in optimizing the problems.

What is claimed is:

1. A medical disease feature selection method based on an improved salp swarm algorithm, the method comprises the following steps:
Step S1, acquiring a microarray gene data set of medical diseases, marking a line number of the microarray gene data set of the medical diseases as m and a column number thereof as n, wherein the microarray gene data set of the medical diseases, which is obtained, is formed by arranging m*n gene feature data according to m lines and n columns; partitioning the microarray gene data set of the medical diseases into 10 subsets randomly by using a 10-cross validation function, wherein a line number of each of the 10 subsets is greater than or equal to 1 and column numbers are n; and selecting one subset from the 10 subsets as a validation set, the rest of the subsets being training sets;
Step S2, defining a female salp population Y, wherein a size of the female salp population Y being M=20, i.e., there are M individuals in the female salp population Y, and each of the M individuals in the female salp population Yis respectively represented by a data matrix formed by arranging n dimensionality values in one line and n columns; and then performing initialized assignment to each of the dimensionality values of each individual in the female salp population Y by using a random number between 0 and 1 to obtain 0th of the female salp population Y°;
Step S3, setting a global optimum fitness value is best, performing initialized assignment of best to positive infinity, setting a global optimal individual to be a bestposition, and initially setting the bestposition as a data matrix [0, 0, 0, . . . , 0] with one line and n columns;
Step S4, setting a maximum time of an iteration of the female salp population T=50, setting an iterative time variable t and initially setting t as 1;
Step S5, performing a t^{th} iteration on the female salp population, an iteration process specifically including:
Step S5.1, converting each dimensionality value of each individual in a (t−1)^{th} generation female salp population Y^{t-1} into 0 or 1 via transfer functions shown in formulae (1)-(2) to obtain a t^{th} generation binary salp population Bt;
$${B}_{i , j}^{t} = \{ \begin{matrix}1 , & S ( {Y}_{i , j}^{t - 1} ) \geq r \\ 0 , & S ( {Y}_{i , j}^{t - 1} ) < r\end{matrix} \qquad ( 1 )$$
$$S ( {Y}_{i , j}^{t - 1} ) = \frac{1}{1 + {e}^{- ( {Y}_{i , j}^{t - 1} / 3 )}} , \qquad ( 2 )$$
wherein Y_{i,j}^{t-1} represents a dimensionality value in the j^{th} column of the i^{th} individual in the (t−1)^{th} generation female salp population, i is equal to 1, 2, 3, . . . M, j is equal to 1, 2, 3, . . . n, B_{i,j}^{t} represents a dimensionality value in the j^{th} column of the i^{th} individual in the t^{th} generation binary salp population, r is a random number between 0 and 1 and is generated a random function before operation every time, and e is a natural constant;
Step S5.2, constructing feature subsets of each individual in the (t−1)^{th} generation female salp population, the step S5.2 specifically including: judging whether the dimensionality value of each column in the i^{th} individual in the t^{th} generation binary salp population is 1 or not respectively, if it is 1, selecting a gene feature data in a verification set and the 9 training sets located in the column, and if it is 0, not selecting the gene feature data in the verification set and the 9 training sets located in the column, taking a residual part as the feature subsets of the verification set of the i^{th} individual in the (t−1)^{th} generation female salp population after deleting the gene feature data of all unselected columns in the verification set, and taking the residual part as the feature subsets of the 9 training sets of the i^{th} individual in the (t−1)^{th} generation female salp population after deleting the gene feature data of all the unselected columns in the 9 training sets, thereby obtaining the feature subsets of the verification set of each individual in the (t−1)^{th} generation female salp population and the feature subsets of the 9 training sets;
Step S5.3, calculating fitness values of each individual in the (t−1)^{th} generation female salp population by adopting a formula (3) and a formula (4), sequencing all the individuals in the (t−1)^{th} generation female salp population according to the fitness values from small to large, marking a minimum fitness as bF^{t-1}, and taking the individual with a minimum fitness value as a current optimum individual marked as bP^{t-1};
$$f i {t}_{i}^{t - 1} = ( 1 - a c {c}_{i}^{t} ) \times a + \frac{{L}_{i}^{t}}{n} \times b \qquad ( 3 )$$
$${ac c}_{i}^{t} = \frac{c {c}_{i}^{t}}{c {c}_{i}^{t} + u {c}_{i}^{t}} \times 1 \; 00 \% , \qquad ( 4 )$$
wherein fitᵢ^{t-1} represents a fitness value of the i^{th} individual in the (t−1)^{th} generation female salp population, a represents a classification accuracy weight set as 0.05, b represents an optimum feature selection number weight, a relation between a and b is a+b=1, Lᵢ^{t} represents a total column number with the dimensionality value of 1 in the i^{th} individual in the (t−1)^{th} generation female salp population, acct represents a classification accuracy of the i^{th} individual obtained by a K-nearest algorithm, cc[and uct are obtained by performing a classifying statistical test on data in the feature subset of the verification set and data in the feature subsets of the 9 training sets in the i^{th} individual in the (t−1)^{th} generation female salp population by adopting the K-nearest algorithm, ccᵢ^{t} represents a number of corrected classified data in the feature subsets of the verification set and ucᵢ^{t} represents a number of mistaken classified data in the feature subsets of the verification set;
Step S5.4, updating the dimensionality values from a first individual to the M/2th individual in the t^{th} generation binary salp population Bt by adopting a formula (5) respectively to obtain the first individual to the M/2^{th} individual of the t^{th} generation binary salp population F^{t};
$${F}_{k , j}^{t} = \{ \begin{matrix}b {P}_{j}^{t - 1} + {c}^{t} r {1}^{t} r {2}^{t} \geq 0 \\ b {P}_{j}^{t - 1} - {c}^{t} r {1}^{t} r {2}^{t} < 0\end{matrix} \qquad ( 5 )$$
$${c}^{t} = 2 {e}^{- {( \frac{4 * t}{T} )}^{2}} , \qquad ( 6 )$$
wherein k is equal to 1, 2, 3 . . . , M/2, r1^{t} and r2^{t} is respectively a random number generated by the random function between 0 and 1, c^{t} is a control parameter represented by a formula (6), bPⱼ^{t-1} represents a j^{th} column dimensionality value of the current optimum individual bP^{t-1}, F_{k,j} represents a kth individual the j^{th} column dimensionality value in a t^{th} generation initial salp population Ft and e is the natural constant;
Step S5.5, updating the dimensionality values from the (M/2+1)^{th} individual to the M^{th} individual in the t^{th} generation binary salp population Bt by adopting a formula (7) respectively by means of a self-adapted control parameter to obtain the (M/2+1)^{th} individual to the M^{th} 5 individual of the t^{th} generation binary salp population F^{t}:
$${F}_{d}^{t} = \frac{1}{2} \times cos ( \frac{p i \times t}{2 T} ) \times ( {B}_{d}^{t} + {B}_{d - 1}^{t} ) \qquad ( 7 )$$
wherein d is equal to M/2+1, M2+2, M2+3, . . . , M, Bt represents the d^{th} individual of the t^{th} generation binary salp population B^{t}, B_{d-1} ^{t} represents the (d−1)^{th} individual in the t^{th} generation binary salp population B^{t}, F_{d}^{t} represents the d^{th} individual in the t^{th} generation initial salp population F^{t}, pi refers to circularity ratio and cos represents a cosine function;
Step S5.6, calculating the fitness values of each individual in the t^{th} generation initial salp population Ft by adopting a method same with those in the Steps 5.1-5.3, sequencing all the individuals in the t^{th} generation initial salp population Ft according to the fitness values from small to large, and marking the individual with the minimum fitness value as firt, the individual with a secondary minimum fitness value as sect and the individual with a third minimum fitness value as thi^{t};
Step S5.7, exploring and developing the t^{th} generation initial salp population Ft by adopting formulae (8)-(16) based on an elite grey wolf ruling policy to obtain a t^{th} generation intermediate salp population G^{t};
$$d i {s}_{j}^{f i r , t} = \vert 2 r {3}^{t} \times f i {r}_{j}^{t} - {F}_{i , j}^{t} \vert \qquad ( 8 )$$
$${G}_{i , j}^{f i r , t} = f i {r}_{j}^{t} - {A}^{t} \times d i {s}_{j}^{f i r , t} \qquad ( 9 )$$
$${A}^{t} = 2 {β}^{t} \times r {4}^{t} - {β}^{t} \qquad ( 10 )$$
$${β}^{t} = 2 - \frac{2 t}{T} \qquad ( 11 )$$
$$di {s}_{j}^{s e c , t} = \vert 2 r {3}^{t} \times s e {c}_{j}^{t} - {F}_{i , j}^{t} \vert \qquad ( 12 )$$
$${G}_{i , j}^{s e c , t} = s e {c}_{j}^{t} - {A}^{t} \times d i {s}_{j}^{s e c , t} \qquad ( 13 )$$
$$di {s}_{j}^{t h i , t} = \vert 2 r {3}^{t} \times t h {i}_{j}^{t} - {F}_{i , j}^{t} \vert \qquad ( 14 )$$
$${G}_{i , j}^{t h i , t} = t h {i}_{j}^{t} - {A}^{t} \times d i {s}_{j}^{t h i , t} \qquad ( 15 )$$
$${G}_{i , j}^{t} = \frac{{G}_{i , j}^{f i r , t} + {G}_{i , j}^{s e c , t} + {G}_{i , j}^{t h i , t}}{3} , \qquad ( 16 )$$
wherein r3^{t} and r4^{t} are respectively random numbers generated by the random function between 0 and 1, A^{t} and β^{t} are vector coefficients, firⱼ^{t} represents the j^{th} column dimensionality value of the individual with the minimum fitness value in the t^{th} generation initial salp population F^{t}, secⱼ^{t} represents the j^{th} column dimensionality value of the individual with the secondary minimum fitness value in the t^{th} generation initial salp population F^{t}, thiⱼ^{t} represents the j^{th} column dimensionality value of the individual with the third minimum fitness value in the t^{th} generation initial salp population F^{t}, F_{i,j}^{t} represents the j^{th} column dimensionality value of the i^{th} individual in the t^{th} generation initial salp population F^{t}, and G_{i,j}^{t} represents the j^{th} column dimensionality value of the i^{th} individual in the t^{th} generation intermediate salp population G^{t};
Step S5.8, calculating the fitness values of the t^{th} generation intermediate salp population Gt by adopting the method same with those in the Steps S5.1-5.3, combining the M individuals in the t^{th} generation initial salp population Ft and the M individuals in the t^{th} generation intermediate salp population G^{t}, sequencing totally 2M individuals according to the fitness values from small to big, selecting the M individuals with smaller fitness values, and arranging the M individuals randomly as the t^{th} iteration to obtain the t^{th} generation salp populationY^{t}; and
Step S5.9, comparing the minimum fitness value of the t^{th} generation salp population Y^{t} with the global optimum fitness value best, updating best by adopting the minimum fitness value if the minimum fitness value is smaller than the global optimum fitness value best and taking the individual corresponding to the minimum fitness value as a global optimum individual bestposition, and keeping the global optimum individual best position with the global optimum fitness value best invariable if the minimum fitness value is not smaller than the global optimum fitness value best and finishing the t^{th} iteration; and
Step S6, judging whether a current value of t is equal to T or not, if not, updating the value of t by adopting the current value of t plus 1, and then returning to the Step S5 for next an iteration; if it is equal to T, finishing the iteration process, determining columns with the dimensionality values of 1 from the first column to the nth column of the global optimum individual bestposition, which is current, and extracting the gene feature data in the microarray gene data set of the medical diseases in these columns correspondingly to form a selection data set, wherein the selection data set obtained at the time is ae dimensionality-reducing gene feature data set of the medical diseases.
